SEQUENCE LISTING (6 sequences)

SEQ ID NO: 1 - DNA, 20 bp, Artificial Sequence
  PCR Primer
tgatcgccgggttctgccag

SEQ ID NO: 2 - DNA, 25 bp, Artificial Sequence
  PCR Primer
catcatgacagaaaaccccaccacg

SEQ ID NO: 3 - DNA, 22 bp, Artificial Sequence
  PCR Primer
cgccgggttctgccagtcgttg

SEQ ID NO: 4 - DNA, 29 bp, Artificial Sequence
  PCR Primer
gcgctgtgatgtgatccaggatctcattc

SEQ ID NO: 5 - DNA, 22 bp, Artificial Sequence
  PCR Primer
gccgggttctgccagtcgttgc

SEQ ID NO: 6 - DNA, 21 bp, Artificial Sequence
  PCR Primer
gcgatctgtccggtttcccgc

(19) United States
(12) Patent Application Publication
(10) Pub. No.: US 2008/0206743 A1
(43) Pub. Date: Aug. 28, 2008

(54) Rapid and Specific Detection of Enterobacter Sakazakii

(76) Inventor: Mark Barbour, Kingsport, TN (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 10/555,504

(22) PCT Filed: May 12, 2004
(86) PCT No.: PCT/US04/15066

§ 371 Date: Apr. 23, 2008

Related U.S. Application Data
(60) Provisional application No. 60/471,132, filed on May 16, 2003.

(51) Int. Cl. C12Q 1/68; C07H 21/00

(52) U.S. Cl. 435/6; 536/24.32

(57) ABSTRACT

The present invention provides a method for specifically detecting pathogenic Enterobacter sakazakii in a complex sample. The complex sample can be a food sample, water sample, or selectively enriched food matrix. The method of detection may utilize PCR amplification with, or without, an internal positive control, and appropriate primer pairs. The reagents necessary to perform the method can be supplied as a kit and/or in tablet form.

### FIELD OF INVENTION

The field of invention relates to a rapid method for detection of Enterobacter sakazakii bacteria, oligonucleotide molecules and reagents and kits useful therefor, and in particular, to a PCR-based method for detection.

### BACKGROUND OF INVENTION

Enterobacter sakazakii (E. sakazakii) is a gram-negative rod-shaped bacterium within the family Enterobacteriaceae. Also previously known as “yellow-pigmented Enterobacter cloacae,” Enterobacter sakazakii was first associated with cases of neonatal meningitis in 1958. Since then, cases of meningitis, septicemia, and necrotizing enterocolitis, due to E. sakazakii, have been reported.

Most documented cases of E. sakazakii infection have involved infants, although reports of infections in adults have also been reported. Fatality rates have varied, but in some cases the rates have been reported to be as high as 80 percent.

While a reservoir for E. sakazakii is unknown, a growing number of reports suggest a role for powdered milk-based infant formulas as a vehicle for infection. For a review of such case studies, see, e.g., Donald H. Burr, “Microbial Detection of Enterobacter sakazakii: Food and Clinical, A White Paper,” delivered at the Contaminants And Natural Toxicants Subcommittee Meeting, Enterobacter Sakazakii Contamination in Powdered Infant Formula, Mar. 18-19, 2003.

It is desirable, therefore, to have a test for the rapid detection of Enterobacter sakazakii.

### SUMMARY OF INVENTION

A method for detecting the presence of Enterobacter sakazakii in a sample, the method comprising: performing PCR amplification of the sample using a primer pair selected from the group consisting of SEQ ID NOs:1 and 2, SEQ ID NOs:3 and 4, or SEQ ID NOs:5 and 6, to produce a PCR amplification result; and examining the PCR amplification result, whereby a positive PCR amplification result indicates the presence of Enterobacter sakazakii in the sample. Preferably, the examining step comprises a melting curve analysis. This method may further comprise a step of preparing the sample for PCR amplification prior to the step of performing PCR amplification. Preferably, the preparing step comprises at least one of the following processes: (1) bacterial enrichment, (2) separation of bacterial cells from the sample, (3) cell lysis, and (4) total DNA extraction. The sample preferably comprises a food or water sample, and even more preferably, a selectively enriched food matrix.

An isolated polynucleotide for detection of Enterobacter sakazakii, consisting essentially of a nucleic acid sequence comprising SEQ ID NO:1, SEQ ID NO:2, SEQ ID NO:3, SEQ ID NO:4, SEQ ID NO:5, or SEQ ID NO:6.

A kit for detection of Enterobacter sakazakii in a sample, or a tablet for use in performance of PCR, comprising: at least one pair of PCR primers selected from the group consisting of SEQ ID NOs:1 and 2, SEQ ID NOs:3 and 4, or SEQ ID NOs:5 and 6; and a thermostable DNA polymerase. Preferably, a kit for detection of Enterobacter sakazakii in a sample comprises the aforementioned tablet.

### SUMMARY OF THE SEQUENCES

SEQ ID NO:1 is the nucleotide sequence of a 5′ primer to a region of the Enterobacter sakazakii genome that will specifically detect Enterobacter sakazakii in a polymerase chain reaction with bacterial DNA and SEQ ID NO:2.

SEQ ID NO:2 is the nucleotide sequence of a 3′ primer to a region of the Enterobacter sakazakii genome that will specifically detect Enterobacter sakazakii in a polymerase chain reaction with bacterial DNA and SEQ ID NO:1.

SEQ ID NO:3 is the nucleotide sequence of a 5′ primer to a region of the Enterobacter sakazakii genome that will specifically detect Enterobacter sakazakii in a polymerase chain reaction with bacterial DNA and SEQ ID NO:4.

SEQ ID NO:4 is the nucleotide sequence of a 3′ primer to a region of the Enterobacter sakazakii genome that will specifically detect Enterobacter sakazakii in a polymerase chain reaction with bacterial DNA and SEQ ID NO:3.

SEQ ID NO:5 is the nucleotide sequence of a 5′ primer to a region of the Enterobacter sakazakii genome that will specifically detect Enterobacter sakazakii in a polymerase chain reaction with bacterial DNA and SEQ ID NO:6.

SEQ ID NO:6 is the nucleotide sequence of a 3′ primer to a region of the Enterobacter sakazakii genome that will specifically detect Enterobacter sakazakii in a polymerase chain reaction with bacterial DNA and SEQ ID NO:5.

### BRIEF DESCRIPTION OF THE DRAWINGS

FIG. 1 shows the process of melting curve analysis. The change in fluorescence of the target DNA is captured during melting. Mathematical analysis of the negative of the change of the log of fluorescence divided by the change in temperature plotted against the temperature results in the graphical peak known as a melting curve.

FIG. 2 shows a representative melting curve analysis for an Enterobacter sakazakii-positive sample in which an internal positive control is added in accordance with a preferred embodiment of the instant application.

### DETAILED DESCRIPTION OF PREFERRED EMBODIMENTS

The disclosure of each reference set forth herein is incorporated by reference in its entirety.

The present invention includes a method to detect, identify, and differentiate pathogenic Enterobacter sakazakii based on the amplification of, or hybridization to, a region of the Enterobacter sakazakii genome.

Oligonucleotides of the instant invention have been developed for the detection and identification of Enterobacter sakazakii.

These oligonucleotides may be used as primers for polymerase chain reaction (PCR) amplification. These oligonucleotide primers would also be useful for other nucleic acid amplification methods such as the ligase chain reaction (LCR) (Backman et al., 1989, EP 0 320 308; Carrino et al., 1995, J. Microbiol. Methods 23: 3-20); nucleic acid sequence-based amplification (NASBA), (Carrino et al., 1995, supra); and self-sustained sequence replication (3SR) and ‘Q replicase amplification’ (Pfeffer et al., 1995 Veterinary Res. Comm., 19: 375-407).

Oligonucleotides of the instant invention also may be used as hybridization probes. Hybridization using DNA probes has been frequently used for the detection of pathogens in food, clinical and environmental samples, and the methodology are generally known to one skilled in the art. It is generally recognized that the degree of sensitivity and specificity of probe hybridization is lower than that achieved through the previously described amplification techniques.

Both amplification-based and hybridization-based methods using the oligonucleotides of the invention may be used to confirm the identification of Enterobacter sakazakii in enriched or even purified culture. A preferred embodiment of the instant invention comprises (1) culturing a complex sample mixture in a non-selective growth media to resuscitate the target bacteria, (2) releasing total target bacterial DNA and, (3) subjecting the total DNA to amplification protocol with a primer pair of the invention.

The amplified nucleic acids may be identified by, for example, gel electrophoresis, nucleic acid probe hybridization, fluorescent end point measurement, or melting curve analysis, as will be explained in more detail below.

This invention allows for the rapid and accurate determination of whether a sample contains Enterobacter sakazakii.

Primers/Oligonucleotides

Three primer sets, PS1 (consisting of two oligonucleotides having the sequences of SEQ ID NO:1 and SEQ ID NO:2), PS2 (consisting of two oligonucleotides having the sequences of SEQ ID NO:3 and SEQ ID NO:4), and PS3 (consisting of two oligonucleotides having the sequences of SEQ ID NO:5 and SEQ ID NO:6) were designed based on internal sequence analysis of a region of the Enterobacter sakazakii genome. Blast searches of the NCBI database revealed no significant sequence homologies to genes of known function. A primer design program (Primer Express®, Applied Biosystems) was used that eliminates detrimental primer configurations such as primer dimers or hairpins, while maintaining specificity for each target organism.

As mentioned above, each of SEQ ID NOs:1-6 may also be used as hybridization probes.

Sample Preparation

The oligonucleotides and methods according to the instant invention may be used directly with any suitable clinical or environmental samples, without any need for sample preparation. In order to achieve higher sensitivity, and in situations where time is not a limiting factor, it is preferred that the samples be pre-treated, and that pre-amplification enrichment is performed.

The minimum industry standard for the detection of food-borne bacterial pathogens is a method that will reliably detect the presence of one pathogen cell in 25 g of food matrix as described in Andrews et al., 1984, “Food Sample and Preparation of Sample Homogenate”, Chapter 1 in Bacteriological Analytical Manual, 8th Edition, Revision A, Association of Official Analytical Chemists, Arlington, Va. In order to satisfy this stringent criterion, enrichment methods and media have been developed to enhance the growth of the target pathogen cell in order to facilitate its detection by biochemical, immunological or nucleic acid hybridization means. Typical enrichment procedures employ media that will enhance the growth and health of the target bacteria and also inhibit the growth of any background or non-target microorganisms present. For example, the U.S. Food and Drug Administration (FDA) has set forth a protocol for enrichment of samples of infant formula to be tested for Enterobacter sakazakii. See “Isolation and Enumeration of Enterobacter sakazakii from Dehydrated Powdered Infant Formula,” U.S. Food and Drug Administration, Center for Food Safety and Applied Nutrition, July 2002, Revised August 2002.

Selective media have been developed for a variety of bacterial pathogens and one of skill in the art will know to select a medium appropriate for the particular organism to be enriched. A general discussion and recipes of non-selective media are described in the FDA Bacteriological Analytical Manual. (1998) published and distributed by the Association of Analytical Chemists, Suite 400, 2200 Wilson Blvd, Arlington, Va. 22201-3301.

After selective growth, a sample of the complex mixtures is removed for further analysis. This sampling procedure may be accomplished by a variety of means well known to those skilled in the art. In a preferred embodiment, 5 μl of the enrichment culture is removed and added to 200 μl of lysis solution containing protease. The lysis solution is heated at 37° C. for 20 min followed by protease inactivation at 95° C. for 10 min as described in the BAX® System User's Guide, Qualicon, Inc., Wilmington, Del.

Amplification Conditions

A skilled person will understand that any generally acceptable PCR conditions may be used for successfully detecting the target Enterobacter sakazakii bacteria using the oligonucleotides of the instant invention, and depending on the sample to be tested and other laboratory conditions, routine optimization for the PCR conditions may be necessary to achieve optimal sensitivity and specificity. Optimally, they achieve PCR amplification products from all of the intended specific targets while giving no PCR product for other, non-target species.

In a preferred embodiment, the following reagents and cycling conditions may be used. Forty-five microliters of lysate added to a PCR tube containing one BAX® reagent tablet (manufactured by Qualicon, Inc., Wilmington, Del.), the tablet containing Taq DNA polymerase, deoxynucleotides, SYBR® Green (Molecular Probes, Eugene, Oreg.), and buffer components, and 5 microliters of primer mix, to achieve a final concentration in the PCR of 0.150 micromoles for each primer. PCR cycling conditions: 94° C., 2 min initial DNA denaturation, followed by 38 cycles of 94° C., 15 seconds and annealing/extension at 70° C. for 3 minutes.

Homogenous PCR

Homogenous PCR refers to a method for the detection of DNA amplification products where no separation (such as by gel electrophoresis) of amplification products from template or primers is necessary. Homogeneous detection of the present invention is typically accomplished by measuring the level of fluorescence of the reaction mixture in the presence of a fluorescent dye.

In a preferred embodiment, DNA melting curve analysis is used, particularly with the BAX® System hardware and reagent tablets from Qualicon Inc. The details of the system are given in U.S. Pat. No. 6,312,930 and PCT Publication Nos. WO 97/11197 and WO 00/66777, each of which is hereby incorporated by reference in its entirety.

Melting Curve Analysis

Melting curve analysis detects and quantifies double stranded nucleic acid molecule (“dsDNA” or “target”) by monitoring the fluorescence of the amplified target (“target amplicon”) during each amplification cycle at selected time points.

As is well known to the skilled artisan, the two strands of a dsDNA separate or melt, when the temperature is higher than its melting temperature. Melting of a dsDNA molecule is a process, and under a given solution condition, melting starts at a temperature (designated T_{MS} hereinafter), and completes at another temperature (designated T_{ME} hereinafter). The familiar term, Tm, designates the temperature at which melting is 50% complete.

A typical PCR cycle involves a denaturing phase where the target dsDNA is melted, a primer annealing phase where the temperature optimal for the primers to bind to the now-single-stranded target, and a chain elongation phase (at a temperature T_{E}) where the temperature is optimal for DNA polymerase to function. According to the present invention, T_{MS} should be higher than T_{E}, and T_{ME} should be lower (often substantially lower) than the temperature at which the DNA polymerase is heat-inactivated. Melting characteristics are effected by the intrinsic properties of a given dsDNA molecule, such as deoxynucleotide composition and the length of the dsDNA.

Intercalating dyes will bind to double stranded DNA. The dye/dsDNA complex will fluoresce when exposed to the appropriate excitation wavelength of light, which is dye dependent and the intensity of the fluorescence may be proportionate to concentration of the dsDNA. Methods taking advantage of the use of DNA intercalating dyes to detect and quantify dsDNA are known in the art. Many dyes are known and used in the art for these purposes. The instant methods also take advantage of such relationship. An example of such dyes includes intercalating dyes. Examples of such dyes include, but are not limited to, SYBR Green-I®, ethidium bromide, propidium iodide, TOTO®-1 {Quinolinium, 1-1′-[1,3-propanediylbis [(dimethyliminio)-3,1-propanediyl]]bis[4-[(3-methyl-2(3H)-benzothiazolylidene) methyl]]-, tetraiodide}, and YoPro® {Quinolinium, 4-[(3-methyl-2(3H)-benzoxazolylidene)methyl]-1-[3-(trimethylammonio)propyl]-, diiodide}. Most preferred dye for the instant invention is a non-asymmetrical cyanide dye such as SYBR Green-I®, manufactured by Molecular Probes, Inc. (Eugene, Oreg.).

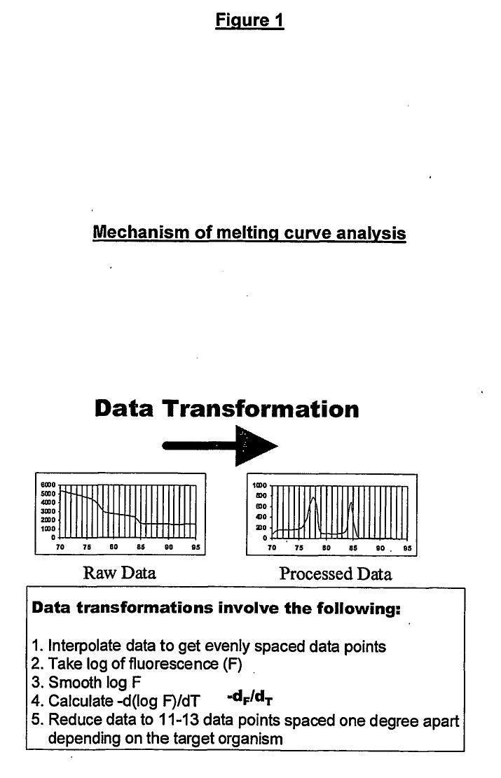

Melting curve analysis is achieved by monitoring the change in fluorescence while the temperature is increased. When the temperature reaches the T_{MS} specific for the PCR amplicon, the dsDNA begins to denature. When the dsDNA denatures, the intercalating dye dissociates from the DNA and fluorescence decreases. Mathematical analysis of the negative of the change of the log of fluorescence divided by the change in temperature plotted against the temperature results in the graphical peak known as a melting curve (FIG. 1).

The data transformation process shown in FIG. 1 involves the following:

1. Interpolate data to get evenly spaced data points

2. Take a log of the fluorescence (F)

3. Smooth log F

4. Calculate-d(log F)/dT

5. Reduce data to 11-13 data points spaced one degree apart (depending on the target organism).

The instant detection method can be used to detect and quantify target dsDNAs, from which the presence and level of target organisms can be determined. The instant method is very specific and sensitive. The fewest number of target dsDNA detectable is between one and 10.

Internal Positive Control

In a preferred embodiment, a PCR amplification composition contains an internal positive control. The advantages of an internal positive control contained within the PCR reaction have been previously described (U.S. Pat. No. 6,312,930 and PCT Application No. WO 97/11197, each of which is hereby incorporated by reference in its entirety) and include: (i) the control may be amplified using a single primer; (ii) the amount of the control amplification product is independent of any target DNA contained in the sample; (iii) the control DNA can be tableted with other amplification reagents for ease of use and high degree of reproducibility in both manual and automated test procedures; (iv) the control can be used with homogeneous detection, i.e., without separation of product DNA from reactants; and (v) the internal control has a melting profile that is distinct from other potentially produced amplicons in the reaction.

Control DNA will be of appropriate size and base composition to permit amplification in a primer directed amplification reaction. The control DNA sequence may be obtained from the target bacteria, or from another source, but must be reproducibly amplified under the same conditions that permit the amplification of the target amplicon DNA.

The control reaction is useful to validate the amplification reaction. Amplification of the control DNA occurs within the same reaction tube as the sample that is being tested, and therefore indicates a successful amplification reaction when samples are target negative, i.e. no target amplicon is produced. In order to achieve significant validation of the amplification reaction a suitable number of copies of the control DNA must be included in each amplification reaction.

Instrumentation

According to a preferred embodiment, the BAX® System (DuPont Qualicon, Wilmington, Del.) and melting curve analysis are used.

Kits and Reagent Tablets

Any suitable nucleic acid replication composition can be used for the instant invention. A typical PCR amplification composition contains for example, dATP, dCTP, dGTP, dTTP, target specific primers and a suitable polymerase. If the nucleic acid composition is in liquid form, suitable buffers known in the art may be used (Sambrook, J. et al. 1989, Molecular Cloning: A Laboratory Manual, Second Edition, Cold Spring Harbor Laboratory Press).

Alternatively, if the composition is contained in a tableted reagent, then typical tabletting reagents may be included such as stabilizers and the like. Preferred tabletting technology is set forth in U.S. Pat. Nos. 4,762,857 and 4,678,812, each of which is hereby incorporated by reference in its entirety.

A preferred kit for detection of Enterobacter sakazakii comprises (a) at least one pair of PCR primers selected from the group consisting of (i) SEQ ID NOs:1 and 2, (ii) SEQ ID NOs:3 and 4, and (iii) SEQ ID NOs:5 and 6; and (b) a thermostable DNA polymerase.

A preferred tablet comprises (a) at least one pair of PCR primers selected from the group consisting of (i) SEQ ID NOs:1 and 2, (ii) SEQ ID NOs:3 and 4, and (iii) SEQ ID NOs:5 and 6; and (b) a thermostable DNA polymerase. Even more preferably, a kit for detection of Enterobacter sakazakii comprises the foregoing preferred tablet.

Replication compositions may be modified depending on whether they are designed to be used to amplify target DNA or the control DNA. Replication compositions that will amplify the target DNA (test replication compositions) may include (i) a polymerase (generally thermostable), (ii) a primer pair capable of hybridizing to the target DNA and (iii) necessary buffers for the amplification reaction to proceed. Replication compositions that will amplify the control DNA (positive control, or positive replication composition) may include (i) a polymerase (generally thermostable) (ii) the control DNA; (iii) at least one primer capable of hybridizing to the control DNA; and (iv) necessary buffers for the amplification reaction to proceed.

In some instances it may be useful to include a negative control replication composition. The negative control composition will contain the same reagents as the test composition but without the polymerase. The primary function of such a control is to monitor spurious background fluorescence in a homogeneous format when the method employs a fluorescent means of detection.

### EXAMPLES

The present invention is further defined in the following Examples. It should be understood that these Examples, while indicating preferred embodiments of the invention, are given by way of illustration only.

### General Methods and Materials Used in the Examples

Materials and Methods suitable for the maintenance and growth of bacterial cultures are well known in the art. Techniques suitable for use in the following Examples may be found in Manual of Methods for Genus Bacteriology (Phillipp Gerhardt, R. G. E. Murray, Ralph N. Costllow, Eugene W. Nester, Willis A. Wood, Noel R. Krieg and G. Briggs Phillips, eds), American Society for Microbiology, Washington, D.C. (1994) or Thomas D. Brock in Biotechnology: A Textbook of Industrial Microbiology, Second Edition (1989) Sinauer Associates, Inc., Sunderland, Mass. or Bacteriological Analytical Manual. 6th Edition, Association of Official Analytical Chemists, Arlington, Va. (1984).

The medium used to grow the Enterobacter sakazakii strains and comparative non-target strains was Brain Heart Infusion broth (BHI) obtained from BBL (Becton-Dickenson). Samples of Enterobacter sakazakii strains were obtained from cultures grown overnight in BHI broth then diluted to approximately 10⁶ cfu/ml in 0.1% peptone water. Samples of the comparative non-target strains were enriched in BHI at approximately 10⁹ cfu/ml.

Primers (SEQ ID NOs:1-6) were prepared by Sigma-Genosys, The Woodlands, Tex.

The reagents that were used in the PCR were from BAX® System Reagent Tablet Kits (DuPont Qualicon, Wilmington, Del.) and include SYBR® Green (Molecular Probes, Eugene, Oreg.), Taq DNA Polymerase (Applied Biosystems, Foster City, Calif.), deoxynucleotides (Roche Diagnostics, Indianapolis, Ind.), and buffer (EM Science, New Jersey).

All PCR reactions were carried out using a standard BAX® System (DuPont Qualicon, Wilmington, Del.).

The meaning of abbreviations is as follows: “h” means hour(s), “min” means minute(s), “sec” means second(s), “d” means day(s), “mL” means milliliters.

### Examples 1-3

### Amplification of Enterobacter sakazakii Specific DNA Fragments

Three primer sets (PS1, PS2, PS3, Table 1) were designed based on internal sequence analysis of a region of the Enterobacter sakazakii genome. Blast searches of the NCBI database revealed no significant sequence homologies to genes of known function. A primer design program (Primer Express®, Applied Biosystems) was used that eliminates detrimental primer configurations such as primer dimers or hairpins, while maintaining specificity for each target organism.

TABLE 1

| Primer set | SEQ ID NOs |
|---|---|
| PS1 | 1 and 2 |
| PS2 | 3 and 4 |
| PS3 | 5 and 6 |

The three primer sets were run under the standard BAX® system PCR cycling conditions at various primer concentrations (typical range 0.05-0.25 μM) to determine the optimal primer concentration for the reaction, which was 0.25 μM.

The following cycling conditions were tested with the above mentioned primer sets: 94° C., 2 min initial DNA denaturation, followed by 38 cycles of 94° C., 15 seconds and annealing/extension at 70° C. for 3 minutes.

Multiple E. sakazakii strains and non-target strains were tested. Non-target strains were not tested in Example 3 (PS3).

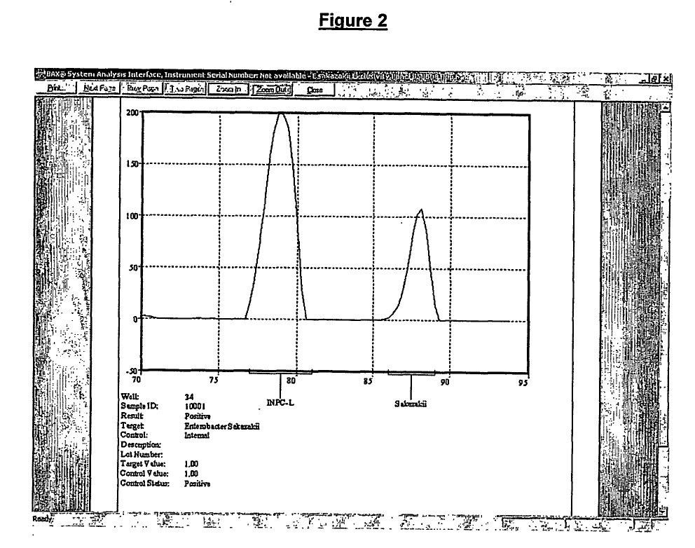

The determination of a positive PCR was achieved with DNA melting curve analysis as mentioned above. A positive reaction for E. sakazakii resulted in the appearance of a melting curve peak at approximately 87-88° C. FIG. 2 shows a representative melting curve analysis for an Enterobacter sakazakii-positive sample in which the internal positive control was added. The internal positive control melts out at approximately 77-78° C., which is clearly distinguishable from the Enterobacter sakazakii amplicons, which have a melting point curve peak at approximately 87-88° C.

The complete results are set forth in Table 2 (PS1, Example 1), Table 3 (PS2, Example 2), and Table 4 (PS3, Example 3).

TABLE 2
Results from PCR with Primer Set PS1 (Example 1)

| | Total | | |
|---|---|---|---|
| | No. of Strains | No. of Positive | No. of Negative |
| Sample | Tested | PCR Product | PCR Product |
| E. sakazakii | 55 | 55 | 0 |
| E. cloacae | 5 | 0 | 5 |
| E. aerogenes | 3 | 0 | 3 |
| E. agglomerans | 5 | 0 | 5 |
| E. hormaichei | 1 | 0 | 1 |
| E. intermedium | 1 | 0 | 1 |
| E. amnigenus | 1 | 0 | 1 |

TABLE 3
Results from PCR with Primer Set PS2 (Example 2)

| | Total No. of | No. of Positive | No. of Negative |
|---|---|---|---|
| Sample | Strains Tested | PCR Product | PCR Product |
| E sakazakii | 55 | 54 | 1 |
| E. cloacae | 4 | 0 | 4 |
| E. agglomerans | 2 | 0 | 2 |
| E. aerogenes | 1 | 0 | 1 |
| E. hormaichei | 1 | 0 | 1 |
| E. amnigenus | 1 | 0 | 1 |
| E. entermedius | 1 | 0 | 1 |
| Esherichia | 1 | 0 | 1 |
| adecarboxylata | | | |

TABLE 4
Results from PCR with Primer Set PS3 (Example 3)

| | Total No. of Strains | No. of Positive | No. of Negative |
|---|---|---|---|
| Sample | Tested | PCR Product | PCR Product |
| E. sakazakii | 55 | 55 | 0 |

What is claimed is:

1. A method for detecting the presence of Enterobacter sakazakii in a sample, the method comprising:
(a) performing PCR amplification of the sample using a primer pair selected from the group consisting of:
(i) SEQ ID NOs:1 and 2,
(ii) SEQ ID NOs:3 and 4, or
(iii) SEQ ID NOs:5 and 6,
to produce a PCR amplification result; and
(b) examining the PCR amplification result,
whereby a positive PCR amplification result indicates the presence of Enterobacter sakazakii in the sample.

2. The method of claim 1, wherein said examining step comprises a melting curve analysis.

3. The method of claim 1, further comprising a step of preparing the sample for PCR amplification prior to said step (a).

4. The method of claim 3, wherein said preparing step comprises at least one of the following processes: (1) bacterial enrichment, (2) separation of bacterial cells from the sample, (3) cell lysis, and (4) total DNA extraction.

5. The method of claim 1, wherein said step (a) comprises performing PCR amplification of the sample using the primer pair (i) SEQ ID NOs:1 and 2.

6. The method of claim 1, wherein the sample comprises a food or a water sample.

7. The method of claim 1, wherein the sample comprises a selectively enriched food matrix.

8. An isolated polynucleotide for detection of Enterobacter sakazakii, consisting essentially of a nucleic acid sequence comprising SEQ ID NO:1, SEQ ID NO:2, SEQ ID NO:3, SEQ ID NO:4, SEQ ID NO:5, or SEQ ID NO:6.

9. The isolated polynucleotide of claim 8, wherein said nucleic acid sequence comprises SEQ ID NO:1 or SEQ ID NO:2.

10. A kit for detection of Enterobacter sakazakii in a sample, the kit comprising:
(a) at least one pair of PCR primers selected from the group consisting of:
(i) SEQ ID NOs:1 and 2,
(ii) SEQ ID NOs:3 and 4, or
(iii) SEQ ID NOs:5 and 6; and
(b) a thermostable DNA polymerase.

11. A tablet for use in performance of PCR comprising:
(a) at least one pair of PCR primers selected from the group consisting
(i) SEQ ID NOs:1 and 2,
(ii) SEQ ID NOs:3 and 4, or
(iii) SEQ ID NOs:5 and 6; and
(b) a thermostable DNA polymerase.

12. A kit for detection of Enterobacter sakazakii in a sample, comprising the tablet of claim 11.
